(19) United States
(12) Patent Application Publication
(10) Pub. No.: US 2013/0035539 A1
(43) Pub. Date: Feb. 7, 2013

(54) SYSTEM AND METHOD FOR TREATING HAIR LOSS

(71) Applicant: ANDREW KORNSTEIN, FAIRFIELD, CT (US)

(72) Inventor: ANDREW KORNSTEIN, FAIRFIELD, CT (US)

(21) Appl. No.: 13/567,540

(22) Filed: Aug. 6, 2012

Related U.S. Application Data
(60) Provisional application No. 61/515,570, filed on Aug. 5, 2011.

(51) Int. Cl. A61N 2/04; A61B 18/18

(52) U.S. Cl. 600/14; 606/33

(57) ABSTRACT

This invention generally relates to an electromagnetic treatment apparatus and a method for using same to achieve modification of cellular and tissue growth, repair, and maintenance, and tissue function by application of electromagnetic information. Particularly, the invention relates to using pulsing electromagnetic fields (PEMF) of very low energy to enhance growth and repair of cells and tissues involved in hair growth and regeneration. More particularly, this invention provides for methods, which are directed to treating hair thinning and/or loss by promoting the maintenance, growth, and restoration of hair by delivery of electromagnetic signals to a target tissue, such as scalp tissue. The electromagnetic signals promote hair cell maintenance, growth, and restoration by modulating biochemical processes that regulate hair cells, such as prostaglandin levels, resulting in stimulation or enhancement of growth, proliferation of hair cells, and or limiting/eliminating hair loss.

### PRIORITY CLAIM

This application claims priority to U.S. Provisional Application No. 61/515,570, filed Aug. 5, 2011, the entirety of which is hereby incorporated by reference.

### BACKGROUND

This invention generally relates to an electromagnetic treatment apparatus and a method for using same to achieve modification of cellular and tissue growth, repair, and maintenance, and tissue function by application of electromagnetic signals. Particularly, the invention relates to using pulsing electromagnetic fields (PEMF) of very low energy to enhance growth, maintenance, and repair of cells and tissues involved in hair growth and regeneration and also to limit or eliminate hair loss.

More particularly, this invention provides for methods and apparatuses, which are directed to treating hair thinning and/or loss by promoting the maintenance, growth, and restoration of hair by delivery of electromagnetic signals to a target tissue, such as scalp tissue. The electromagnetic signals promote hair cell maintenance, growth, and restoration by modulating biochemical processes that regulate hair cells, resulting in stimulation or enhancement of growth and/or proliferation of hair cells, in addition to limiting or eliminating hair loss. In a specific embodiment, the invention pertains to use of a self-contained apparatus that emits time varying magnetic fields that are configured to promote maintenance, growth, and restoration of hair, by a number of purported mechanisms, one being by affecting the initial steps of production and/or release of growth factors, cytokines, and/or prostaglandins, such as the step of ion-ligand interaction, for example calcium interaction with calmodulin.

Hair Loss Disorders

Hair loss disorders, which include hair thinning and loss, are extremely prevalent, affecting an estimated 20-25% of the population. While more common amongst adult males, hair loss disorders also affect women and children. Although most hair loss disorders are not, in themselves, damaging to the health of an individual, many hair loss disorder sufferers report associated psychological problems, such as anxiety and depression.

There are numerous causes of hair thinning and/or loss. Advancing age is associated with hair thinning and/or loss due, in part, to hormonal, biochemical, and inflammatory effects, as well as reduced blood supply. In men, this is known as androgenic alopecia (AGA). Alopecia greata is an autoimmune condition where the body's immune system attacks its own hair follicles. Hair root infection can cause hair loss due to excessive bacterial growth in tiny oil glands of the hair root. Some allergic reactions can cause hair loss by triggering enhanced production by germinal cells of cytokines that inhibit hair growth. Hyper- or hypo-thyroidism can cause areas of hair to thin, become brittle, and then fall out. Additionally, radiation affects the gene or genes required for cell growth and division at the hair papilla and bulb, resulting in hair loss. Chemotherapy treatment for cancer of various types is well known to result in hair loss. Some medications, including some heart and blood pressure medications, thyroid medications, and cholesterol-lowering drugs, are also frequent causes of hair loss. Other causes of hair loss include improper care, diet, stress, trauma, ringworm, Demodex Folliculorum, Ichthyosiform Erhythroderma, Keratosis Follicularis Spinulosa Decalvans, Leprosy, Progeria, Siemens Syndrome, Spastic paraplegia, neuropathy, poikiloderma, anorexia nervosa, chronic anorexia, mental health disorders, and disease affecting general nutritional intake.

Because of the widespread prevalence of hair loss disorders and their numerous and varied causes, there are correspondingly numerous and varied ways in which to treat hair loss disorders in order to maintain, grow, and restore hair. However, to date, there is no known safe, reliable, reproducible, and non-invasive (with least potential side effects) method for treating idiopathic alopecia.

Plastic surgery hair implantation techniques involve the process of removing plugs of bald scalp, filling the defect with plugs of hair roots taken from hair-bearing areas, stretching the hairy scalp to increase surface area, removing and stretching of the expanded hair scalp, serial excision, and pulling the adjacent hair-bearing areas of scalp close to the bald area. These types of treatments are expensive, painful, and may require repeated procedures to restore the scalp hair.

Steroids are also used to treat hair loss disorders. For example, people suffering from alopecia greata are often treated with steroid injections in the scalp. One major drawback to this treatment is that the steroids are systemically absorbed, which may lead to give rise to numerous problems and side effects associated with steroid exposure.

There are numerous drugs designed to treat hair loss disorders as well. In particular, three medications—Minoxidil (Rogaine®), Finasteride (Propecia®, Proscar®) and Latisse® (bimatroprost ophthalmic solution)—have been approved by the FDA for treatment of hair loss and/or as growth accelerants. They may be used prophylactically to avert hair loss or to increase hair growth.

Minoxidil is available without prescription and is applied topically. Side effects of Minoxidil include itching, skin rash, headaches, dizzy spells, irregular heartbeat, chest pain, and decreased sexual ability or desire. Finasteride, taken orally, treats mild to moderate male pattern hair loss. Side effects of Finasteride include breast enlargement (gynecomastia) and tenderness, skin rash, lip swelling, abdominal pain, back pain, decreased sex drive and semen, impotence, diarrhea, dizziness, and headaches. Latisse®, bimatroprost ophthalmic solution, is a prostaglandin derivative that has been approved for growth of eye lash hair. While these drug treatments may be successful in some individuals, the degrees of efficacy of such treatments vary greatly and are often accompanied by undesirable side effects.

Pulsed Electromagnetic Field (PEMF) Therapy

Pulsed electromagnetic field (PEMF) therapy is a reparative technique, which employs electrical energy to direct a series of magnetic pulses through tissue, whereby each magnetic pulse induces a tiny electrical signal that stimulates cellular repair. Pulsed electromagnetic energy has long been used to promote healing in humans. For example, PEMF has been shown to have physiological effects on tissue repair and growth; healing soft tissue wounds; suppressing inflammatory responses; and relieving pain.

There is a growing body of clinical evidence that non-invasive, non-pharmacological PEMF can have physiological effects on inflammation and tissue repair. In fact, PEMF devices have been approved by the U.S. Food and Drug Administration for the relief of post-operative pain and edema. The Centers for Medicare and Medicaid Services (CMS) has approved payment for in-hospital treatment of wound healing.

Cellular studies have shown a demonstrable effect of weak low frequency electromagnetic fields on both signal transduction pathways and growth factor production. EMF delivery has been shown to induce the secretion of growth factors after a short and trigger-like duration. Additionally, ion-ligand binding processes at a cell membrane are generally considered an initial EMF target pathway structure. Numerous studies examining the effects of PEMF on bone repair have shown an upregulation in the synthesis of growth factors, such as Insulin-like Growth Factors (IGF) and Transforming Growth Factor (TGF) in osteoblasts, in a manner dependent on the binding of calcium to calmodulin.

A meta-analysis of randomized clinical trials using PEMF on soft tissues and joints revealed that PEMF was effective in accelerating healing of skin wounds and in the treatment of pain associated with connective tissue injury and joint-associated soft tissue injury. Another study demonstrated that PEMF, adjunctive to standard of care, can provide pain control with a non-invasive modality and reduce morbidity due to pain medication after breast augmentation surgery. A related study showed that PEMF therapy significantly reduced post-operative pain in breast reduction patients, by a mechanism that involved manipulating the dynamics of interleukin-1 beta (IL-1β) in the wound bed by means of a PEMF effect on the calmodulin-dependent nitric oxide signaling, which, in turn, could impact the speed and quality of wound repair.

In addition, studies have shown that PEMF may regulate prostaglandin levels. One study describes PEMF inhibiting the process of neutrophil production if IL-1β, which may upregulate inducible nitric oxide synthase activity, which leads to release of nitric oxide, which results in induction of cyclooxygenase-2, and which results in increasing levels of prostaglandins. Thus, this study demonstrates that one effect of PEMF is the modulation of prostaglandin levels. Of interest one study has demonstrated that prostaglandin D₂ (PGD₂) inhibits hair growth by promoting hair loss.

Studies have also demonstrated that PEMF supports endothelial cell growth and promotes neovascularization and angiogenesis. It is believed that PEMF exerts these effects on blood vessels by changing the cellular plasma membrane potential at the cellular level, encouraging a calcium flux that may stimulate a cellular response. In support of that, PEMF has been shown to accelerate calcium interaction with calmodulin, which may result in the production of growth factors.

Much of the art pertaining to both electromagnetic radiation and hair actually relates to hair removal, i.e., not hair maintenance, growth, and restoration. For example, U.S. Pat. No. 6,280,438 is directed to a method for removing hair by the application of intense, wide area, pulsed electromagnetic energy. Additionally, U.S. Pat. No. 5,885,273 discloses a method for removing hair that includes producing a plurality of pulses of incoherent electromagnetic energy, which is filtered in accordance with the color of the hair being removed. U.S. Patent Publication No. US 2009/0240243, publication of U.S. patent application Ser. No. 12/388,348, discloses a hair-removal device using pulsed electromagnetic radiation. Thus, much of the art pertaining to the effect of electromagnetic radiation on hair teaches away from the use of electromagnetic radiation for hair maintenance, growth, and restoration.

Despite the existence of numerous and varied options for treatment of hair thinning and hair loss, there remains a need in the art for a safe, reproducible, reliable, and non-invasive means to treat hair loss disorders by promoting maintenance, growth, and restoration of hair.

### DESCRIPTION OF THE EMBODIMENTS

This invention generally relates to an electromagnetic treatment apparatus and a method for using same to achieve modification of cellular and tissue growth, repair, and maintenance, and function by application of electromagnetic signals. Particularly, the invention relates to using pulsing electromagnetic fields (PEMF) of very low energy to enhance growth and repair of cells and tissues involved in hair growth and regeneration and also to limit or eliminate hair loss.

More particularly, this invention provides for methods and apparatuses, which are directed to treating hair thinning and/or loss by promoting the maintenance, growth, and restoration of hair by delivery of electromagnetic signals to a target tissue, such as scalp tissue. The electromagnetic signals promote hair cell maintenance, growth, and restoration by their direct modulation of biochemical processes that regulate hair cells, resulting in stimulation or enhancement of growth and/or proliferation of hair cells and/or limiting or eliminating hair loss. In a specific embodiment, the invention pertains to use of a self-contained apparatus that emits time varying magnetic fields that are configured to promote maintenance, growth, and restoration of hair, by affecting the initial steps of production and/or release of growth factors, cytokines, and/or prostaglandins, such as the step of ion-ligand interaction, for example calcium interaction with calmodulin.

There are numerous parameters associated with a treatment apparatus that delivers PEMF. Such parameters include, but are not limited to, wave shape, frequency, pulse rate, burst rate, burst repetition rate, peak signal amplitude, induced electric field, duration, and others. These parameters may be altered or adjusted to achieve a particular configuration that will elicit the desired bioeffect on a molecule, cell, tissue, or organ.

PEMF parameters may be configured to promote interaction of ions with regulatory molecules, such as calcium binding to calmodulin. Use of this calcium-calmodulin pathway is based upon its known roles in acceleration of tissue repair, for example promotion of hair maintenance, growth, and restoration. Growth factors such as platelet derived growth factor (PDGF), fibroblast growth factor (FGF), and epidermal growth factor (EGF) are all involved in appropriate stages of hair maintenance, growth, and restoration. Moreover, angiogenesis and neovascularization are also integral to hair maintenance, growth, and restoration and may also be modulated. It is also thought that levels of prostaglandins (some which promote hair growth and some which inhibit hair growth) may be important in regulating hair growth and/or hair loss. The loss of hair may also be inflammatory in nature. All of these effects are dependent on the interaction of calcium with calmodulin. Thus, a waveform that utilizes a pathway that promotes the interaction of calcium with calmodulin can have physiologically significant bioeffect on hair maintenance, growth, and restoration. It is believed that in this way, PEMF encourages hair follicles to advance from a quiescent resting phase to a growth phase and/or limits or eliminates hair loss.

Without being bound by any theory or mechanism of action, it is believed that one way in which PEMF promotes hair maintenance, growth, and restoration is by inhibiting prostaglandin D₂ (PGD₂). One study describes PEMF inhibiting the process of neutrophil production if IL-1β, which upregulates inducible nitric oxide synthase activity, which leads to release of nitric oxide, which results in induction of cyclooxygenase-2, and which results in increasing levels of prostaglandins. Thus, this study demonstrates that one effect of PEMF is a direct alteration of prostaglandin levels. Furthermore, one study has demonstrated that PGD₂ inhibits hair growth. Therefore, one major effect of PEMF exposure is the reduction of PGD₂, which results in promotion of hair growth and/or limiting or eliminating hair loss.

Furthermore, Minoxidil (Rogaine®), a well-known drug for treatment of hair loss, may elicit its effects by increasing the production of a prostaglandin known to be down-regulated in bald scalps versus haired scalps, i.e., Prostaglandin E₂ (PGE₂). It is thought that efforts to enhance PGE₂ and to inhibit PGD₂ signaling may result in promotion of hair maintenance, growth, and restoration. For example, PEMF treatment may be used in combination with Minoxidil treatment to enhance PGE₂ and to inhibit PGD₂ signaling. The effect of PEMF and Minoxidil treatment may be synergistic. For example, PEMF causes vasodilation and increased local blood flow, thereby enabling enhanced absorption of topically applied Minoxidil to achieve increased concentrations where it may optimally affect PGE₂.

While not being bound by any theory or mechanism of action, it is believed that at least one way in which PEMF promotes hair maintenance, growth, and restoration is by the modulation of local expression of growth factors, such as PDGF, FGF, and EGF. Indeed it is well known that hair growth is modulated by numerous endogenous substances (see Table 1). One way in which growth factor expression may be modulated is via a pathway involving nitric oxide (NO) and cyclic guanosine monophosphate (cGMP). Studies have shown that PEMF induces vasodilatation through NO release. Subsequent NO and cGMP activity leads to the production of other growth and healing factors and the decrease or increase in levels of various prostaglandins. Some growth factors that are induced by this mechanism include Vascular Endothelial Growth Factor (VEGF), which modulates angiogenesis; Fibroblast Growth Factor (FGF), which modulates collagen and granulation; and Transforming Growth Factor-Beta (TGF-β), which modulates remodeling. Some prostaglandins influenced by NO release may include Prostaglandin D₂ (PGD₂) and/or Prostaglandin E₂ (PGE₂).

TABLE 1
Endogenous Substances That Affect Hair Growth

| | | Effect on |
|---|---|---|
| Substance | Site of Action | Hair Growth |
| Basic Fibroblast Growth Factor | Dermal papilla cells | Increase (H) |
| (bFGF) | | |
| Platelet-Derived Growth Factor | Dermal papilla cells | Increase (H) |
| (PDGF) | | |
| Transforming Growth Factor-Beta | Dermal papilla cells | Decrease (H) |
| (TGF-β) | | |
| Interleukin-1-Alpha (IL-1-α) | Hair matrix cells | Decrease (H) |
| Fibroblast Growth Factor Type 5 | Hair matrix cells | Decrease (H) |
| (FGF5) | | |
| Epidermal Growth Factor (EGF) | Hair matrix cells | Decrease (H) |
| Keratinocyte Growth Factor (KGF) | Hair matrix cells | Increase (R) |
| Insulin-Like Growth Factor-I (IGF- | Hair matrix cells | Increase (H) |
| I) | | |
| Substance P | Unknown | Increase (M) |
| Parathyroid Hormone (PTH) | Unknown | Decrease (M) |
| 1,25-Dihydroxyvitamin D3 | Unknown | Concentration- |
| (1,25/OH/D3) | | Dependent |
| | | (increase (H) |
| | | at low |
| | | concentration; |
| | | and |
| | | decrease (H) at |
| | | high |
| | | concentration) |

Table 1: Table 1 shows endogenous substances which affect hair growth. The species studied is noted in parenthetical adjacent to the listed effect (H: Human; R: Rat; and M: Mouse).

While not being bound by any theory or mechanism of action, it is believed that one other possible way in which PEMF promotes hair maintenance, growth, and restoration is by eliciting local anti-inflammatory effects. It has been demonstrated previously, that PEMF promotes the interaction of calcium with calmodulin in a voltage-dependent fashion, which can occur in a matter of milliseconds. Additionally, the subsequent binding of the calcium-calmodulin complex with endothelial nitric oxide synthase (eNOS) catalyzes the release of NO. Nitric oxide induced in this voltage-dependent fashion can elicit anti-inflammatory effects, by promoting increased blood and lymph flow locally.

Without being bound by any theory or mechanism of action, it is also believed that there may be more than one way in which PEMF promotes hair maintenance, growth, and restoration. It is possible that one or more effects work in collaboration to promote hair maintenance, growth, and restoration.

In one embodiment, the apparatus that delivers PEMF is self-contained, lightweight, and portable. In some cases, the apparatus may also be disposable. The apparatus is preferably safe for home use, so that individuals may use the method on their own. In another embodiment, a miniature control circuit is coupled to a generating device, such as an electric coil via a connector. The miniature control circuit is designed to configure waveforms that produce physiologically beneficial results when applied to hair.

In a specific embodiment according to the present invention, the parameters of the frequency output are as follows: carrier frequency of between 1-50 MHz, such as 27.12 MHz±150 KHz; burst width and rate of 2 ms burst width at 2 Hz; peak power output of 0.5 Watt; average power (measured over 1 sec.) of 2 milli-watts; and a standard load of 50 Ohm.

A waveform configured using a specific embodiment according to the present invention may be applied to a hair target pathway structure such as ions for a total exposure time of under 1 minute to 240 minutes daily. For example, the exposure time is about 15 minutes twice daily for 4-6 months. Alternatively, the exposure time may be about 15 minutes twice daily for an indefinite period of time. However, other exposure times may be employed.

Waveforms configured by the miniature control circuit are directed to a generating device such as electrical coils via a connector. The generating device delivers a pulsing magnetic field that can be used to provide treatment to scalp and/or hair. The miniature control circuit applies a pulsing magnetic field for a prescribed time and can automatically repeat the application of the pulsing magnetic field for as many applications as are needed in a given time period, for example 10 times a day. The miniature control circuit can be configured to be programmable, applying pulsing magnetic fields for any time repetition sequence.

A specific embodiment can be configured to treat hair by being incorporated into, or by otherwise including, a positioning device, thereby making the unit self-contained. Advantageously, miniature circuitry and ultra lightweight coils allow for convenient use of the apparatus. In this way, hair maintenance, growth, and restoration may be accomplished and enhanced anywhere and at anytime. Additionally, in certain embodiments, the apparatus is preferably placed around the scalp or head region in order to treat patients with hair loss disorders affecting the scalp or head region. Preferably, the apparatus is adjustable, so that it may accommodate the different and varied sizes and shapes of scalps and heads. The apparatus may, for example, be incorporated into an article, such as a hat, so that the apparatus may be worn inconspicuously. The apparatus may further incorporate a disposable battery. Alternatively, the apparatus may incorporate a rechargeable battery.

Another embodiment according to the present invention applies a model to induce a time-varying electric field in a hair target pathway structure, such as ions and ligands (e.g., calcium-calmodulin), comprising about 0.1-100 msec bursts of about 1-100 microsecond rectangular pulses repeating at about 0.1-100 pulses per second. Peak amplitude of the induced electric field is between about 1 μV/cm and about 100 mV/cm, varied according to a modified function, inversely related to frequency. In another embodiment, the apparatus delivers PEMF in 2 ms bursts of 27.12 MHz sinusoidal waves repeating at 2 bursts/second, having a peak magnetic field of 0.05 G, which induces an average electric field of 32±6 mV/cm. In another embodiment, the apparatus delivers PEMF in 65 μsec bursts of 27.12 MHz sinusoidal waves, inducing a 1 G high amplitude peak magnetic field, repeating at 600 bursts per second. In another embodiment, the apparatus delivers PEMF in 1 msec bursts of 27.12 MHz waves at 5 bursts/second, with 0.02 G of peak amplitude. In a related embodiment, the apparatus delivers PEMF in 1 msec bursts of 27.12 MHz waves at 5 bursts/second, with 0.05 G of peak amplitude. In yet another embodiment, the apparatus delivers PEMF in 2 msec bursts of 27.12 MHz repeating at 5 bursts/second with a peak amplitude of 0.05 G. In one embodiment, the apparatus delivers PEMF in 2-20 msec bursts of 27.12 MHz waves having a peak amplitude of 0.1 G. In a related embodiment, the apparatus delivers PEMF in 2-20 msec bursts of 27.12 MHz waves having peak amplitude of 2.0 G. In yet another embodiment, the apparatus delivers PEMF in 2 msec bursts of 27.13 MHz waves repeating at 5 bursts/second with a peak power of 0.05 Gauss.

In a specific embodiment, the claimed invention is directed to a method for the treatment of hair loss for a subject in need thereof, comprising the steps of: (1) obtaining an apparatus comprising a pulsing electromagnetic fields (PEMF) signal generating device configured to deliver PEMF to target tissue; and a control circuit, which configures the parameters of the PEMF signal, wherein the control circuit configures the PEMF signal generating device to (a) deliver PEMF having a carrier frequency of about 1-50 MHz; and (b) to regulate the levels of one or more prostaglandins; and (2) using the apparatus to apply PEMF to target tissue in an area of the body on which hair growth is desired.

In a specific embodiment of the method of the claimed invention, the PEMF regulates the level of one or more prostaglandins, by decreasing the level of Prostaglandin D₂ (PGD₂), which results in a reduction in hair loss.

In a specific embodiment of the method of the claimed invention, the PEMF signal generating device is an electric coil that may be flexible and lightweight. In another embodiment of the method of the claimed invention, the PEMF signal generating device is a conductive thread.

In a specific embodiment of the method of the claimed invention, the control circuit is a miniature control circuit. In a specific embodiment of the method of the claimed invention, the PEMF signaling generating device is coupled to the miniature control circuit via a connector.

In a specific embodiment of method of the claimed invention, the target tissue is scalp tissue. In a specific embodiment, the apparatus is configured to accommodate the scalp or head region. In a related embodiment of the method of the claimed invention, the apparatus is configured to adjustably accommodate a variety of sizes and shapes of scalps and head regions.

In a specific embodiment of the method of the claimed invention, the apparatus is self-contained, lightweight, portable, and safe for home use.

In a specific embodiment of the method of the claimed invention, the apparatus contains a battery. In a related embodiment of the method of the claimed invention, the battery may be a disposable battery or a rechargeable battery.

In a specific embodiment of the method of the claimed invention, the parameters of the PEMF signal are configured to promote the interaction of ions with regulatory molecules within the target tissue. In a related embodiment of the method of the claimed invention, the interaction of ions with regulatory molecules comprises the binding of calcium to calmodulin.

In a specific embodiment of the method of the claimed invention, the carrier frequency is 27.12 MHz.

In a specific embodiment of the method of the claimed invention, the control circuit configures the PEMF generating device to deliver PEMF having a burst width and rate of about 2 ms burst width at about 2 Hz.

In a specific embodiment of the method of the claimed invention, instructions are provided for using the apparatus to promote hair maintenance, growth, and restoration. In a specific embodiment of the method of the claimed invention, the instructions indicate an exposure time for PEMF delivery of about 15 minutes. In a specific embodiment of the method of the claimed invention, the instructions indicate that the apparatus is to be used twice daily. In one embodiment, the instructions for using the apparatus to promote hair growth indicate that the apparatus is to be used for a period of about 4-6 months. In a specific embodiment of the method of the claimed invention, the instructions indicate that the apparatus is to be used for a period longer than 6 months. In a specific embodiment of the method of the claimed invention, the instructions indicate that the apparatus is to be used indefinitely.

In another embodiment of the method of the claimed invention, the method further comprises the step of (3) treating the subject with a different method of treatment for treatment of hair loss for a subject in need thereof. In a related embodiment, the different method of treatment for hair loss is administration of Minoxidil. In yet another related embodiment, the Minoxidil increases the levels of Prostaglandin E₂ (PGE₂). In another embodiment, the combination of PEMF and Minoxidil results in a synergistic effect for treatment of hair loss.

In a specific embodiment of the method of the claimed invention, the hair loss is due to alopecia greata, thereby avoiding steroid absorption and associated side effects, which are often observed in conventional treatment of alopecia greata with steroids. In a specific embodiment of the method of the claimed invention, the hair loss is due to chemotherapy. In a specific embodiment of the method of the claimed invention, hair implantation viability is maintained after a hair transplant.

### EXAMPLES

### Figures

FIG. 1A: A photograph of the part of a scalp from a female patient before (labeled “Pre”) and after (labeled “Post”) treatment with PEMF for two months using the IVIVI SofPulse® device.

FIG. 1B: A photograph of the crown of a scalp from a female patient before (labeled “Pre”) and after (labeled “Post”) treatment with PEMF for two months using the IVIVI SofPulse® device.

FIG. 2A: A photograph of an apical view of a scalp from a male patient before (labeled “Pre”) and after (labeled “Post”) treatment with PEMF for two months using the IVIVI SofPulse® device.

FIG. 2B: A photograph of a frontal view of a scalp from a male patient before (labeled “Pre”) and after (labeled “Post) treatment with PEMF for two months using the IVIVI SofPulse® device.

A group of 12 patients, who were experiencing scalp hair loss disorders, enrolled in a study examining the effects of delivery of PEMF on hair maintenance, growth, and restoration. The parameters of the frequency output of PEMF were as follows: carrier frequency of 27.12 MHz±150 KHz; burst width and rate of 2 ms burst width at 2 Hz; peak power output of 0.5 Watt; average power (measured over 1 sec.) of 2 milli-watts; and a standard load of 50 Ohm.

For purposes of examining the effects of PEMF delivery on hair maintenance, growth, and restoration in this example, patients used the SofPulse® Electroceutical™ System Device (“SofPulse® Device”), produced by IVIVI Health Sciences (San Francisco, Calif.). Patients were subjected to PEMF delivery for two 15 minute treatments a day, for a period of at least 4 months. During treatments, each patient placed a SofPulse® Device on the top of their head and self-administered PEMF treatment. Periodically, patients” scalps were examined grossly to determine the effect of PEMF on scalp hair maintenance, growth, and restoration. Gross examinations of the scalp hair were documented and monitored by photographs and patient evaluations.

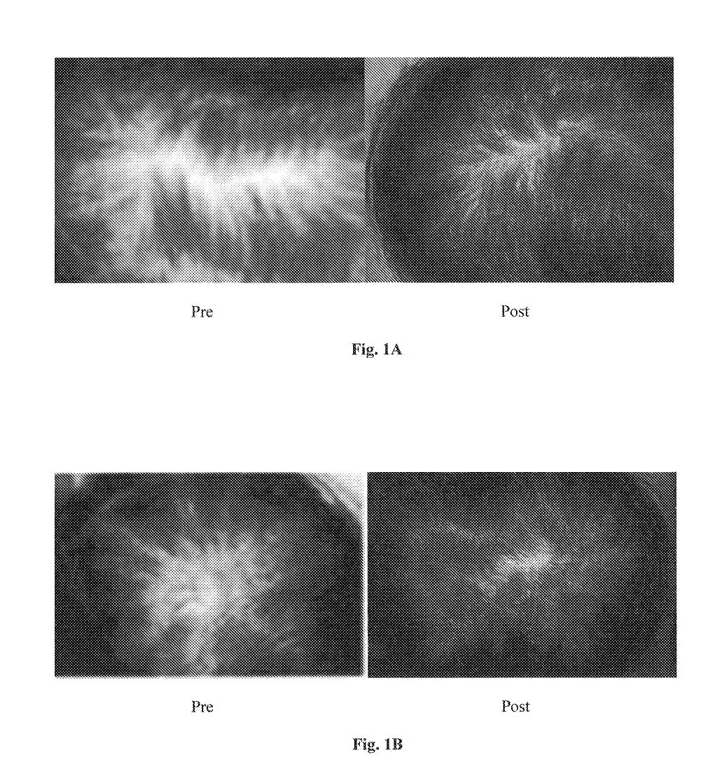

FIG. 1 shows photographs of a female patient before (labeled “Pre”) and after (labeled “Post”) two months of treatment with PEMF delivered to the scalp. FIG. 1A shows the part of the scalp and FIG. 1B shows the crown.

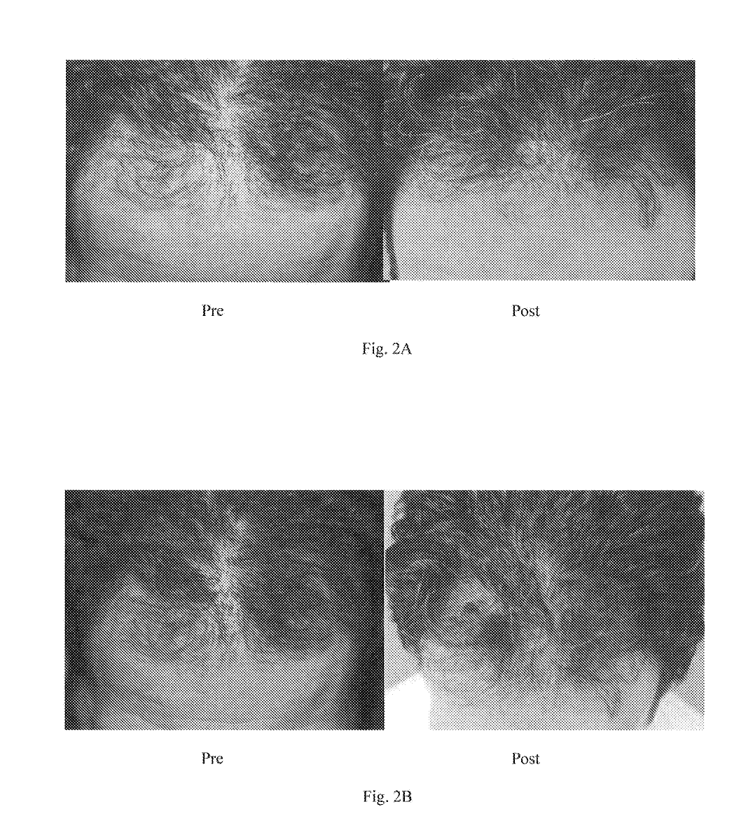

FIG. 2 shows photographs of a male patient before (labeled “Pre”) and after (labeled “Post”) two months of treatment with PEMF delivered to the scalp. FIG. 2A shows an apical view of the scalp and FIG. 2B shows a frontal view of the scalp.

FIGS. 1 and 2 show that after just two months of treatment with PEMF delivered to the scalp, the scalps of patients suffering from hair loss disorders are able to promote hair growth. All other patients enrolled in the study experienced and reported similar hair growth after just a few months of PEMF treatment.

While the present invention has been disclosed with reference to certain embodiments, numerous modifications, alterations, and changes to the described embodiments are possible without departing from the spirit and scope of the present invention, described herein and in the appended claims. Accordingly, it is intended that the present invention not be limited to the described embodiments, but that it has the full scope accorded by law.

What is claimed is:

1. A method for the treatment of hair loss for a subject in need thereof, comprising the steps of:
(a) obtaining an apparatus comprising a pulsing electromagnetic fields (PEMF) signal generating device configured to deliver PEMF to target tissue; and a control circuit, which configures the parameters of the PEMF signal,
wherein the control circuit configures the PEMF signal generating device to (a) deliver PEMF having a carrier frequency of about 1-50 MHz; and (b) to regulate the level of one or more prostaglandins; and
(b) using the apparatus to apply PEMF to target tissue in an area of the body on which hair growth and/or limiting or eliminating hair loss is desired.

2. The method of claim 1, wherein the PEMF regulates the level of one or more prostaglandins, by decreasing the level of Prostaglandin D₂ (PGD₂).

3. The method of claim 2, wherein decreasing level of PGD₂ results in a limiting or elimination of hair loss.

4. The method of claim 1, wherein the PEMF signal generating device is an electric coil.

5. The method of claim 4, wherein the electric coil is flexible and lightweight.

6. The method of claim 1, wherein the PEMF signal generating device is a conductive thread.

7. The method of claim 1, wherein the control circuit is a miniature control circuit.

8. The method of claim 7, wherein the PEMF signal generating device is coupled to the miniature control circuit via a connector.

9. The method of claim 1, wherein the target tissue is scalp tissue.

10. The method of claim 9, wherein the apparatus is configured to accommodate the scalp or head region.

11. The method of claim 10, wherein the apparatus is configured to adjustably accommodate a variety of sizes and shapes of scalps and head regions.

12. The method of claim 1, wherein the apparatus is self-contained, lightweight, portable, and safe for home use.

13. The method of claim 1, wherein the apparatus contains a battery.

14. The method of claim 13, wherein the battery is a disposable battery.

15. The method of claim 13, wherein the battery is a rechargeable battery.

16. The method of claim 1, wherein the parameters are configured to promote the interaction of ions with regulatory molecules within the target tissue.

17. The method of claim 14, wherein the interaction of ions with regulatory molecules comprises the binding of calcium to calmodulin.

18. The method of claim 1, wherein the carrier frequency is 27.12 MHz.

19. The method of claim 1, wherein the control circuit configures the PEMF signal generating device to deliver PEMF having a burst width and a rate of about 2 ms burst width at about 2 Hz.

20. The method of claim 1, further comprising providing instructions for using the apparatus to promote hair growth.

21. The method of claim 20, wherein the instructions for using the apparatus to promote hair growth indicate an exposure time for PEMF delivery of about 15 minutes.

22. The method of claim 1, wherein the instructions for using the apparatus to promote hair growth indicate that the apparatus is to be used twice daily.

23. The method of claim 1, wherein the instructions for using the apparatus to promote hair growth indicate that the apparatus is to be used for a period of about 4-6 months.

24. The method of claim 1, wherein the instructions for using the apparatus to promote hair growth indicate that the apparatus is to be used indefinitely

25. The method of claim 1, further comprising the step of (3) treating the subject with a different method of treatment for treatment of hair loss for a subject in need thereof.

26. The method of claim 25, wherein the different method of treatment for hair loss is administration of Minoxidil.

27. The method of claim 26, wherein the Minoxidil increases the levels of Prostaglandin E₂ (PGE₂).

28. The method of claim 26 or 27, wherein the combination of PEMF and Minoxidil results in a synergistic effect for treatment of hair loss.

29. The method of claim 1, wherein the hair loss is due to alopecia greata.

30. The method of claim 29, wherein steroid absorption and associated side effects are avoided.

31. The method of claim 1, wherein the hair loss is due to chemotherapy treatment.

32. The method of claim 1, wherein hair implantation viability is maintained after a hair transplant.

33. A method for the treatment of hair loss for a subject in need thereof, comprising the steps of:
(a) obtaining an apparatus comprising a pulsing electromagnetic fields (PEMF) signal generating device configured to deliver PEMF to target tissue; and a control circuit, which configures the parameters of the PEMF signal,
wherein the control circuit configures the PEMF signal generating device to (a) deliver PEMF having a carrier frequency of about 1-50 MHz; and (b) to regulate the level of one or more prostaglandins; and
(b) using the apparatus to apply PEMF to target tissue in an area of the body on which hair growth and/or limiting or eliminating hair loss is desired
wherein the PEMF regulates the level of one or more prostaglandins, by decreasing the level of Prostaglandin D₂ (PGD₂), resulting in a limiting or elimination of hair loss.
